(19) United States
(12) Patent Application Publication
(10) Pub. No.: US 2012/0308636 A1
(43) Pub. Date: Dec. 6, 2012

(54) Multi-Layer Antimicrobial Wound Dressing

(75) Inventors: Jo-Wei Huang, Chong-Li City (TW); Mei-Ting Fan Chiang, Chong-Li City (TW)

(73) Assignee: FAR EASTERN NEW CENTURY CORPORATION, Taipei City (TW)

(21) Appl. No.: 13/483,643

(22) Filed: May 30, 2012

(30) Foreign Application Priority Data
May 31, 2011 (TW) 100119043

(51) Int. Cl. A61L 15/44

(52) U.S. Cl. 424/445; 514/55

(57) ABSTRACT

A multi-layer antimicrobial wound dressing includes a wound-contacting layer and an antimicrobial layer. The wound contacting layer includes a biocompatible fibrous matrix that is made of an anionic polymer, and the antimicrobial layer is made of a cationic polymer that is connected to the wound-contacting layer via anionic-cationic interaction between the anionic polymer of the fibrous matrix and the cationic polymer of the antimicrobial layer.

### CROSS-REFERENCE TO RELATED APPLICATION

This application claims priority of Taiwanese application No. 100119043, filed on May 31, 2011.

### BACKGROUND OF THE INVENTION

1. Field of the Invention

The present invention is directed to a multi-layer wound dressing, specifically an antimicrobial wound dressing with high absorption capacity.

2. Description of the Related Art

A conventional absorbent material can be classified into two major categories: the first is an acrylic acid-based material, and the second is a carbohydrate-based material. The acrylic acid-based material is low cost and possesses high absorbency. However, it is not biodegradable by microorganisms and cannot be decomposed naturally by the environment. In addition, since acrylic acid-based material is not biocompatible, it is likely to elicit allergy reaction when used as a wound dressing material. The carbohydrate-based material, e.g., starch, chitosan, sodium alginate, carboxymethyl cellulose, etc., possesses good biocompatibility. However, the absorbency thereof is not ideal. Heavily exuding wounds would cause maceration and infection of tissues surrounding the highly exuding wounds when the carbohydrate-based material is used as a dressing. The macerated tissues may also adversely affect the adhesion of the dressing.

Traditional wound dressings are mostly in the form of a film. In recent years, materials for the wound dressing have evolved into a fibrous structure to increase the surface area that come in contact with wounds, thereby efficiently absorbing exudates from the wounds. U.S. Pat. No. 6,075,177 discloses a wound dressing having a wound-contacting layer composed of carboxymethyl cellulose filaments capable of absorbing at least 15 times its own weight of saline solution. However, such absorbency is still insufficient for a heavily exuding wound. In addition, the wound can be easily infected if the wound dressing does not have any antimicrobial activity.

U.S. Pat. No. 6,458,460 discloses a wound dressing composed of at least two discrete fibers. Both of the fibers are hygroscopic fibers in which one fiber is a discrete modified cellulose gel forming fiber, and the other is alginate discrete gel forming fiber. Since the total surface area of the fibers is larger than that of a film, it exhibits a higher absorbency than films. However, the capacity of cellulose and alginate to absorb fluid is limited and can only absorb less than 20 times of its original weight. Thus, these fibers cannot effectively absorb exudates and lacks antimicrobial activity.

Sliver ions are long known for its antimicrobial activity and have been utilized in medical care products. It is disclosed in U.S. Pat. No. 7,576,255, U.S. Pat. No. 7,329,417 and U.S. Pat. No. 7,323,614 wound dressings having antimicrobial activity by incorporating silver ions. The antimicrobial effect provided by silver ions is acted upon the binding of silver ions to key enzymes of the microorganism which have electron donating functional groups, such as carboxylates, thiol, amines, etc. The binding of silver to these functional groups leads to an irreversible deactivation and death of the microorganisms. However, silver imposes a certain degree of toxicity to humans. In addition, silver is easily detached from the wound dressing, thus lowering the efficacy of antimicrobial effect provided by silver ions.

Accordingly, there is a need in the art to develop a wound dressing that is composed of natural materials, and that have antimicrobial activity and high absorbency.

### SUMMARY OF THE INVENTION

According to the present invention, a multi-layer antimicrobial wound dressing comprises: a wound-contacting layer including a biocompatible fibrous matrix that is made of an anionic polymer; and an antimicrobial layer that is made of a cationic polymer and that is connected to the wound-contacting layer via anionic-cationic interaction between the anionic polymer of the fibrous matrix and the cationic polymer of the antimicrobial layer.

### BRIEF DESCRIPTION OF THE DRAWING

Other features and advantages of the present invention will become apparent in the following detailed description of the preferred embodiments of this invention, with reference to the accompanying drawings, in which:

FIG. 1 is a cross sectional view showing the preferred embodiment of a multi-layer antimicrobial wound dressing of the present invention that comprises a wound-contacting layer and an antimicrobial layer connected to the wound-contacting layer via anionic-cationic interaction.

### DETAILED DESCRIPTION OF THE PREFERRED EMBODIMENTS

The preferred embodiment of a multi-layer antimicrobial wound dressing of the present invention includes a wound-contacting layer and an antimicrobial layer.

The wound-contacting layer includes a biocompatible fibrous matrix that is made of an anionic polymer. The anionic polymer includes an anionic polypeptide. The anionic polypeptide is selected from the group consisting of polyglutamic acid, derivatives of polyglutamic acid, polyaspartic acid, derivatives of polyaspartic acid, and combinations thereof.

The derivatives of each of polyglutamic and polyaspartic acids can be obtained by modifying the ratio of functional groups, such as the ratio of COOH group to COONa group, to thereby obtain derivatives with increased solubility.

The derivatives of polyglutamic acid and polyaspartic acid also include salts thereof, such as magnesium salt, calcium salt, sodium salt, etc.

The biocompatible fibrous matrix has a unit volume weight ranging from 30 mg/cm³ to 220 mg/cm³. When the unit volume weight of the fibrous matrix of the wound-contacting layer is below 30 mg/cm³, the fibrous matrix has low fiber density. Thus, the wound dressing can not attain its function, such as covering the wound completely. When the unit volume weight of the fibrous matrix is greater than 220 mg/cm³, the fibrous matrix has an excessively high fiber density, thereby resulting in over expansion in volume of the wound dressing after absorbing exudates. Preferably, the unit volume weight of the fibrous matrix ranges from 40 mg/cm³ to 200 mg/cm³.

Preferably, the biocompatible fibrous matrix has a thickness ranging from 0.15 cm to 0.4 cm. When the thickness of the wound-contacting layer is less than 0.15 cm, it will be completely covered by the antimicrobial layer, thus adversely affecting the absorbent properties of the wound-contacting layer. When the thickness of the wound-contacting layer is greater than 0.4 cm, the resultant oversized wound dressing after absorbing exudates cannot be conveniently used. More preferably, the biocompatible fibrous matrix has a thickness ranging from 0.2 cm to 0.3 cm.

The antimicrobial layer is in contact with the wound-contacting layer, and is composed of a cationic polymer. The cationic polymer includes cationic polysaccharide. The cationic polysaccharide is selected from the group consisting of chitosan, derivatives of chitosan, and combinations thereof. Examples of derivative of chitosan include N-octyl-O and N-carboxymethyl chitosan. Derivatives of chitosan that are cationic and biocompatible are suitable for the present invention. Preferably, the weight average molecular weight of the cationic polymer ranges from 6,000 to 900,000, more preferably, from 100,000 to 800,000. When the weight average molecular weight is less than 6,000, the antimicrobial effect is low, since the antimicrobial effect is positively correlated to the net positive charge of chitosan. When the weight average molecular weight is greater than 900,000, the cationic polymer has low solubility and will be difficult to process. Moreover, production of the cationic polymer with high molecular weight is hard.

The antimicrobial activity of chitosan depends on the content of protons of the ammonia group (—NH₃⁺) present in chitosan at a low pH. Deacetylation process involves the removal of acetyl group on chitosan and leaving a highly active primary amino group (—NH₂). The active primary amino group (—NH₂) will become the ammonia group (—NH₃⁺) at the low pH. The protons on the ammonia groups would bind to the phospholipids on bacterial cell walls, leading to cell death.

Preferably, the deacetylation degree of chitosan is greater than 70%, more preferably, greater than 85%. When the deacetylation degree is less than 70%, the antimicrobial activity becomes less effective.

The antimicrobial layer containing a cationic polymer is bound to the wound-contacting layer that is composed of an anionic polymer via cationic-anionic interaction. With the cationic-anionic interaction, adhesion between the antimicrobial layer and the wound-contacting layer would be enhanced.

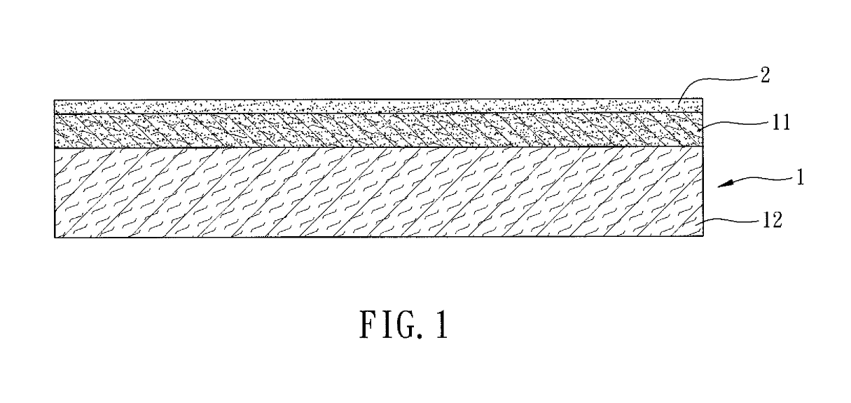

FIG. 1 shows an example of the multi-layer antimicrobial wound dressing including a wound-contacting layer 1 composed of anionic polyglutamic acid and an antimicrobial layer 2 composed of cationic chitosan. The wound-contacting layer 1 includes a base sub-layer 12 and a reacted sub-layer 11 disposed between the base sub-layer 12 and the antimicrobial layer 2. The anionic-cationic interaction between the anionic polyglutamic acid and the cationic chitosan occurs in the reacted sub-layer 11.

The preparation method of the antimicrobial wound dressing includes: (a) providing the biocompatible fibrous matrix as the wound-contacting layer, that is made of the anionic polymer and that has specific unit volume weight and thickness; (b) dissolving an appropriate amount of the cationic polymer in a proper solvent to obtain a cationic polymeric solution; and (c) coating the cationic polymeric solution on the wound-contacting layer. Alternatively, step (c) can be conducted by immersing a part of the wound-contacting layer in the cationic polymeric solution. The resultant product will have a triple-layer structure, i.e., the base sub-layer 12 and the reacted sub-layer 11 of the wound-contacting layer 1 and the antimicrobial layer 2 shown in FIG. 1. Lastly, the product is dried to obtain an integrated piece of the multi-layer antimicrobial wound dressing.

Preferably, the viscosity of the cationic polymeric solution ranges from 200 cp to 4300 cp, more preferably, from 240 cp to 3050 cp. When the viscosity is less than 200 cp, which indicates a low solid content of the cationic polymer, the obtained antimicrobial layer is unlikely to completely cover the biocompatible fibrous matrix, thereby leading to a compromised antimicrobial effect. When the viscosity of the cationic polymeric solution is greater than 4300 cp, this results in a thick solution that is hard to process, and may cause uneven spreading of the viscous cationic polymeric solution on the wound-contacting layer, thereby resulting in wrinkles on the antimicrobial layer.

### EXAMPLES

### Experimental Materials:

This invention will be further described by way of the following examples. However, it should be understood that the following examples are solely intended for the purpose of illustration and should not be construed as limiting the invention in practice.

### <Source of Chemicals>

1. Polyglutamic acid: purchased from Vedan; catalog number: γ-PGA (sodium form).
2. Chitosan: purchased from Aldrich; product name: Poly (D-glucosamine).
3. Acetic acid: purchased from Echo Chemical Co., Ltd; Analytical grade.
4. Malic acid: purchased from Aldrich; purity: ≧99.0%.

### <Preparation of Polyglutamic Acid (PGA) Fibrous Matrix>:

PGA fibers were prepared according to TW Patent Publication No. 201037110. The PGA fibers were subjected to wet-spinning methods to obtain a PGA fibrous matrix. The PGA fibrous matrix, formed by collecting PGA fibers on the rollers, can be made into a desired unit volume weight (mg/cm³) and thickness (cm) by controlling the speed of the roller and collecting duration. The parameters for preparing the PGA fibrous matrix used in the Examples are shown in Table 1.

TABLE 1

| | | |
|---|---|---|
| Roller | Collecting | PGA fibrous matrix |

| | speed | duration | Unit volume | Thickness |
|---|---|---|---|---|
| No. | (rpm) | (min) | weight (mg/cm³) | (cm) |
| 1 | 50 | 30 | 67 | 0.3 |
| 2 | 80 | 30 | 107 | 0.3 |
| 3 | 35 | 20 | 35 | 0.2 |
| 4 | 40 | 15 | 40 | 0.15 |
| 5 | 140 | 30 | 215 | 0.4 |
| 6 | 50 | 30 | 67 | 0.3 |
| 7 | 80 | 30 | 107 | 0.3 |
| 8 | 25 | 20 | 24 | 0.2 |
| 9 | 150 | 30 | 232 | 0.3 |

### Preparation of a Multi-Layer Antimicrobial Wound Dressing

### Example 1

1. A PGA fibrous matrix with a unit volume weight of 67 mg/cm³ and a thickness of 0.3 cm was prepared based on the preparation method mentioned in the section of “Preparation of polyglutamic acid (PGA) fibrous matrix”.
2. Chitosan (deacetylation degree 95%, Mw=350,000) was dissolved in 1% of acetic acid solution to obtain a 1 wt % chitosan solution with a viscosity of 916 cp. Subsequently, the chitosan solution was evenly poured into a Petri dish and then evenly spread in the Petri dish to obtain an even layer of chitosan solution with a height of 1.5 mm.
3. The PGA fibrous matrix obtained in step 1 was then positioned in the Petri dish such that the PGA fibrous matrix was partly immersed in the chitosan solution for ten minutes to obtain a multi-layer semi-product. This semi-product was then neutralized by washing with a sodium hydroxide solution, and was further immersed in pure water (pH=6 to 8) for approximately 30 minutes. Subsequently, the semi-product was dried in an oven at 60° C. for two hours, hence obtaining a multi-layer antimicrobial wound dressing.

### Examples 2 to 9

The preparation methods for the antimicrobial absorbent wound dressing for Examples 2 to 9 were the same as that for Example 1, except that: the parameters for the preparation methods are different and are listed in Table 2.

TABLE 2

| | PGA | Chitosan solution | | | | |
|---|---|---|---|---|---|---|
| | fibrous | Degree of | | | Concentration | |
| | matrix | deacetylation | | | of chitosan | Viscosity |
| | No. | (%) | M_{w} (Dₐ) | Solvent | (wt %) | (cp) |
| Example 1 | 1 | 95 | 350,000 | Acetic acid | 1 | 916 |
| Example 2 | 2 | 98 | 500,000 | Malic acid | 1.5 | 1933 |
| Example 3 | 3 | 95 | 880,000 | Malic acid | 1.2 | 2508 |
| Example 4 | 4 | 90 | 6,800 | Acetic acid | 10 | 223 |
| Example 5 | 5 | 78 | 800,000 | Acetic acid | 1.8 | 3979 |
| Example 6 | 6 | 95 | 6,200 | Malic acid | 15 | 274 |
| Example 7 | 7 | 70 | 400,000 | Malic acid | 1.8 | 2333 |
| Example 8 | 8 | 95 | 800,000 | Malic acid | 1 | 2419 |
| Example 9 | 9 | 98 | 80,000 | Malic acid | 2 | 550 |

### Comparative Examples 1 and 2

The preparation methods for the antimicrobial absorbent wound dressings of Comparative Examples 1 and 2 were the same as Example 1, except that: in Comparative Example 1, the PGA fibrous matrix was substituted by a 5″×8″ fibrous cotton pad (no charge or slight negative charge) with a unit volume weight of 67 mg/cm³ and 0.3 cm thickness (purchased from Ubok Co. Ltd.) and in Comparative Example 2, the Chitosan solution was substituted by a natural antimicrobial solution containing citrus extract (Citrofresh, usually without ionic charge) (purchased from Citrofresh).

### Comparative Example 3

The preparation method for the wound dressing of Comparative Example 3 was the same as that for Example 1 except that: the PGA fibrous matrix was substituted with a PGA film. The PGA film was made as follows. Firstly, a 20% PGA aqueous solution was prepared, and 6 μL of ethylene glycol diglycidyl ether was added to each gram of PGA solution, followed by reaction at 60° C. with stirring at 50 rpm for one hour to form a coating solution. Subsequently, the coating solution was applied on a glass surface between two insulators with a thickness of 0.5 cm, followed by drying in an oven at 60° C. for 12 hours, thereby obtaining a PGA film with a 0.15 cm thickness.

### Antimicrobial Testing

### [Bacterial Culture]

A single colony of Staphylococcus aureus (BCRC Number 15211) was randomly picked from an agar plate, and was placed into a 15 mL centrifuge tube containing 2000 μL of LB broth. Subsequently, the 15 mL centrifuge tube was then vortexed for 10 minutes to disrupt the colony to form a uniformly suspended stock solution. The stock solution was subjected to a 10-fold serial dilution to obtain several diluted solutions (10⁻¹, 10⁻², 10⁻³, 10⁻⁴, and 10⁻⁵-fold). 100 μL of each of the diluted solutions was then inoculated on an agar plate and cultured for 14 to 24 hours at 37° C. Based on the calculated colony forming units (CFU) of the cultivated agar plates, a testing solution with a bacterial concentration of 10⁶˜10⁷ CFU/mL was obtained by diluting an appropriate amount of the stock bacterial solution with sterilized water.

### [Antimicrobial Activity Test]

100 μL of the aforesaid testing solution was inoculated on an agar plate. Meanwhile, the samples obtained from Examples 1 to 7 and Comparative Examples 1 and 2 were each formed into a tablet. The chitosan layer of the tablet was in contact with the agar plate that has the testing solution. The agar plate together with the tablet was cultured for 14 to 24 hours in a 37° C. incubator.

The antimicrobial effect of the tablet is visualized as a zone of clearance expanding concentrically from the tablet on the agar plate. The antimicrobial effect was evaluated by the percentage of bacterial growth, i.e. the area of bacterial colony over the total surface area of the agar plate. 100% of bacterial growth was defined as complete coverage of bacterial colonies on the agar plate. The results are shown in Table 3.

In order to test whether the wound-contacting layer and the antimicrobial layer are strongly adhered, another experiment was conducted as follows: the tablet from Example 1 and Comparative Examples 1 and 2 were washed with distilled water ten times and then subjected to the aforesaid antimicrobial activity test.

TABLE 3

| | Colony growth on | Colony growth on |
|---|---|---|
| | plates with different | plates with different |
| | tablets obtained from | washed tablets from |
| | different examples | different examples |
| Example 1 | ⊚ | ⊚ |
| Example 2 | ⊚ | — |
| Example 3 | ⊚ | — |
| Example 4 | ◯ | — |
| Example 5 | ◯ | — |
| Example 6 | Δ | — |
| Example 7 | Δ | — |
| Comparative | ⊚ | X |
| Example 1 | | |
| Comparative | ⊚ | X |
| Example 2 | | |

Note:
“⊚” indicates no colony formation, suggesting good antimicrobial activity of the tablet; “◯” indicates 1% to 50% bacterial growth, suggesting the tablet has some antimicrobial activity; “Δ” indicates 50% to 99% bacterial growth, suggesting lesser effect of antimicrobial activity, “X” indicates 100% bacterial growth, suggesting there is no antimicrobial activity, “—” indicates no test was performed.

As shown in Table 3, all of Examples 1 to 7 have antimicrobial activity, in which Examples 1 to 3 have superior antimicrobial activity. In Examples 4 and 6, although the chitosan has relatively high deacetylation degree, the relatively low M_{w} made the tablets in these two examples have lower antimicrobial activity than that of Examples 1 and 3. In Examples 5 and 7, since the deacetylation degree is relatively low, the antimicrobial wound dressings have lower antimicrobial activity than that of Examples 1 and 3.

The wound dressing from Example 1 still retained its antimicrobial activity after washing in water for ten times, suggesting the presence of strong adherence of the wound-contacting layer and the antimicrobial layer provided by the cationic-anionic interaction. On the contrary, results from Comparative Examples 1 and 2 suggest that, when the fibrous matrix layer and the antimicrobial layer are not strongly adhered, the antimicrobial layer will be washed out, thus reducing its antimicrobial activity.

### [Absorbency Test]

In the absorbency test, a moisture analyzer (purchased from A&D Co. Ltd.; catalog number: MX-50) was used to determine moisture content of samples before and after immersing in water. The wound dressings in Examples 1 and 2 and Comparative Examples 1 and 3 were weighed before immersing in water. The wound dressings were then immersed in excess water for 30 seconds to reach saturated absorption. Subsequently, the wound dressings were removed from the water and allowed to drain until no water dripped. The weight of each of the wound dressings was recorded after immersion in water. The absorbency rate of the wound dressing was calculated using Formula (I). The results are shown in Table 4.

Absorbency rate=(weight after immersing in water−weight before immersing in water)/weight before immersing in water (I)

TABLE 4

| | | | Comparative | Comparative |
|---|---|---|---|---|
| | Example 1 | Example 2 | Example 1 | Example 3 |
| Absorbency | 92.7 | 125.1 | 8.25 | 1.96 |
| rate | | | | |

As shown in Table 4, the absorbency rate is superior in Examples 1 and 2 when compared to Comparative Examples 1 and 3, suggesting that the PGA fibrous matrix of this invention has a significantly higher absorbency rate than that of fibrous cotton pad (e.g., Comparative Example 1). Data from Comparative Example 3 suggests that the structure of the wound dressing affects water absorbency rate as well. Specifically, the wound dressings with a fibrous structure (e.g., Examples 1 and 2) have a greater surface area than that with a film structure (e.g., Comparative Example 3). The greater the surface area that comes in contact with the exudates, the faster will be removal of the exudates, thus preventing maceration and infection of the wound.

To sum up, the present invention utilizes anionic-cationic interaction of the anionic and cationic polymers to enhance the binding strength of the fibrous matrix of the wound-contacting layer and the antimicrobial layer. Moreover, the materials of the wound dressing are biocompatible and biodegradable, and have antimicrobial activity and good absorbency.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A multi-layer antimicrobial wound dressing, comprising:
a wound-contacting layer including a biocompatible fibrous matrix that is made of an anionic polymer; and
an antimicrobial layer that is made of a cationic polymer and that is connected to said wound-contacting layer via the anionic-cationic interaction between said anionic polymer of said fibrous matrix and said cationic polymer of said antimicrobial layer.

2. The multi-layer antimicrobial wound dressing of claim 1, wherein said anionic polymer includes an anionic polypeptide and said cationic polymer includes a cationic polysaccharide.

3. The multi-layer antimicrobial wound dressing of claim 2, wherein said anionic polypeptide is selected from the group consisting of polyglutamic acid, derivatives of polyglutamic acid, polyaspartic acid, derivatives of polyaspartic acid, and combinations thereof.

4. The multi-layer antimicrobial wound dressing of claim 2, wherein said cationic polysaccharide is selected from the group consisting of chitosan, derivatives of chitosan, and combinations thereof.

5. The multi-layer antimicrobial wound dressing of claim 1, wherein said fibrous matrix has a unit volume weight ranging from 30 mg/cm³ to 220 mg/cm³.

6. The multi-layer antimicrobial wound dressing of claim 5, wherein the unit volume weight ranges from 40 mg/cm³ to 200 mg/cm³.

7. The multi-layer antimicrobial wound dressing of claim 1, wherein said fibrous matrix has a thickness ranging from 0.15 cm to 0.4 cm.

8. The multi-layer antimicrobial wound dressing of claim 7, wherein the thickness ranges from 0.2 cm to 0.3 cm.

9. The multi-layer antimicrobial wound dressing of claim 1, wherein said cationic polymer has a weight average molecular weight ranging from 6,000˜900,000.

10. The multi-layer antimicrobial wound dressing of claim 9, wherein the weight average molecular weight ranges from 100,000˜800,000.

11. The multi-layer antimicrobial wound dressing of claim 4, wherein said cationic polymer has a deacetylation degree greater than 70%.

12. The multi-layer anti-microbial wound dressing of claim 11, wherein the degree of deacetylation is larger than 85%.

13. The multi-layer antimicrobial wound dressing of claim 1, wherein said antimicrobial layer is obtained by applying a solution containing said cationic polymer to a surface of said wound-contacting layer.

14. The multi-layer antimicrobial wound dressing of claim 13, wherein said solution containing said cationic polymer has a viscosity ranging from 200 cp-4300 cp.
